(19) United States
(12) Patent Application Publication
(10) Pub. No.: US 2022/0259641 A1
(43) Pub. Date: Aug. 18, 2022

(54) AMPLIFICATION ASSAY FOR THE DETECTION OF ANAPLASMA PHAGOCYTOPHILUM

(71) Applicants: THE HENRY M. JACKSON FOUNDATION FOR THE ADVANCEMENT OF MILITARY MEDICINE, INC., BETHESDA, MD (US); THE UNITED STATES OF AMERICA AS REPRESENTED BY THE SECRETARY OF THE NAVY, Silver Spring, MD (US)

(72) Inventors: Chien-Chung CHAO, North Potomac, MD (US); Wei-Mei CHING, Silver Spring, MD (US); Le JIANG, Clarksville, MD (US)

(21) Appl. No.: 17/627,254

(22) PCT Filed: Jul. 17, 2020
(86) PCT No.: PCT/US2020/042470

§ 371 Date: Jan. 14, 2022

Related U.S. Application Data
(60) Provisional application No. 62/875,179, filed on Jul. 17, 2019.

(51) Int. Cl. C12Q 1/689; C12Q 1/6883

(52) U.S. Cl. CPC C12Q 1/689; C12Q 1/6883

(57) ABSTRACT

Methods, primers, and kits for detecting Anaplasma phagocytophilum (A. phagocytophilum) by amplification of a multi-copy DNA target sequence found within the msp2 gene of A. phagocytophilum are disclosed. Methods for treating A. phagocytophilum infections, including tick-borne fever and human granulocytic anaplasmosis are also disclosed.

### CROSS-REFERENCE TO RELATED APPLICATIONS

This application claims priority to U.S. Provisional Application No. 62/875,179, filed Jul. 17, 2019, the contents of which are hereby incorporated by reference in its entirety.

### STATEMENT REGARDING FEDERALLY SPONSORED RESEARCH

This invention was made with government support under HT9404-13-0024 awarded by the Uniformed Services University of the Health Sciences. The government has certain rights in this invention.

### SEQUENCE LISTING

This application contains a Sequence Listing which has been submitted electronically in ASCII format and is hereby incorporated by reference in its entirety. Said ASCII copy, created on 15 Jul. 2020, is named HJF_553-19_SEQLIST_ST25.txt and is 3,279 bytes in size.

### FIELD OF THE INVENTION

The present application generally relates to methods, primers, and kits for detecting Anaplasma phagocytophilum (A. phagocytophilum) by amplification of a multicopy DNA target sequence found within the msp2 gene of A. phagocytophilum.

### BACKGROUND OF THE INVENTION

A. phagocytophilum is an obligate intracellular Gram-negative bacterium that can be transmitted to humans and animals mainly through Ixodes ticks present in the northern hemisphere (Stuen et al. 2013 Front Cell Infect Microbiol 3:31). Its infection causes tick-borne fever (TBF) in domestic animals and human granulocytic anaplasmosis (HGA) in human patients. As a multi-host pathogen, A. phagocytophilum puts significant economic burden on livestock production and increases health risks for humans and their pets as well.

Clinical diagnosis of HGA is challenging as many patients present with nonspecific symptoms and signs including fever, malaise, headache, and myalgia (Bakken et al. 2015 Infect Dis Clin North Am 29:341-355). This often delays antibiotic treatment, predominantly doxycycline, which is most effective during the early course of the infection. Traditionally, peripheral blood smears are examined microscopically and the presence of morulae in the cytoplasm of neutrophils can be used for diagnosis during the first week of illness (Rand et al. 2014, Am J Clin Pathol 141:683-686). However, this method might be error-prone in cases of low level of bacteremia or due to other inclusions or cytoplasmic granules. Serology-based clinical tests, such as immunofluorescent assay (IFA), have been useful, but they require the presence of Anaplasma-specific antibodies, which are not detectable until the second week after infection. Furthermore, cross-reactions with other Anaplasma species or closely-related bacterial species, such as Ehrlichia chaffeensis, are possible. Another drawback of the aforementioned methods is that they do not offer direct pathogen detection in invertebrates, such as its vectors for prevalence studies.

DNA-based molecular detection has long been used for identification of Anaplasma species and offers much higher levels of sensitivity and specificity. For example, DNA sequences within rrs (Massung et al. 1998 J Clin Microbiol 36:1090-1095), msp2 (Courtney et al. 2004 J Clin Microbiol 42:3164-3168), and msp4 (de la Fuente et al. 2005 J Clin Microbiol 43:1309-1317) genes have been used for conventional or real-time polymerase chain reaction (PCR) assays for A. phagocytophilum detection. However, PCR-based direct pathogen detection requires well-trained technicians and expensive equipment, which are usually not readily available in remote areas.

Recombinase polymerase amplification (RPA) assay was developed as a novel method to efficiently amplify DNA at isothermal conditions (between 37 to 42° C.), thus providing a simple, rapid alternative for nucleic acid detection (Piepenburg et al. 2006 PLoS Biol 4:e204). It has been successfully used to detect bacterial pathogen DNA (Kersting et al. 2014 Mikrochim Acta 181:1715-1723; Liu et al. 2017 J Dairy Sci 100:7016-7025) and when coupled with a reverse transcriptase, it can also effectively detect RNA viruses (Abd El Wahed et al. 2013 PLoS ONE 8:e71642; Amer et al. 2013 J Virol Methods 193:337-340). There remains, however, the need for a highly sensitive and specific assay for detecting A. phagocytophilum. In particular, there remains the need for point-of-care diagnostic methods and tools, as well as methods for vector surveillance and epidemiologic studies, particularly in resource-constrained regions where other A. phagocytophilum detection methods are not readily available.

### SUMMARY OF THE INVENTION

In a first aspect, the application relates to a method of detecting the presence of Anaplasma phagocytophilum (A. phagocytophilum) in a sample comprising (a) amplifying a 171 base pair (bp) target DNA sequence encoded by SEQ ID NO: 1, or a fragment thereof, within the msp2 gene in the A. phagocytophilum genome; and (b) detecting the amplification products of step (a); wherein the detection of amplification products in step (b) indicates the presence of A. phagocytophilum in the sample.

In another aspect, the application relates to a method of diagnosing tick-borne fever (TBF) in an animal comprising detecting the presence of A. phagocytophilum in a sample from said animal, said method comprising (a) amplifying a 171-bp target DNA sequence encoded by SEQ ID NO: 1, or a fragment thereof, within the msp2 gene in the A. phagocytophilum genome; and (b) detecting the amplification products of step (a); wherein the detection of said amplification products indicates a positive diagnosis of TBF in said animal.

In yet another aspect, the application relates to a method of diagnosing human granulocytic anaplasmosis (HGA) in a human comprising detecting the presence of A. phagocytophilum in a sample from said human, said method comprising a) amplifying a 171-bp target DNA sequence encoded by SEQ ID NO: 1, or a fragment thereof, within the msp2 gene in the A. phagocytophilum genome; and (b) detecting the amplification products of step (a); wherein the detection of said amplification products indicates a positive diagnosis of HGA in said human.

In yet another aspect, the application relates to primers designed for use in the amplification step of the methods of the present application. In certain aspects, the fragment of the 171-bp target DNA is selected from the group consisting of nucleotide fragments ranging from about 50 to about 100 nucleotides in length or at least about 100 nucleotides in length, found within SEQ ID NO: 1, and in certain aspects, the fragment of the 171-bp target DNA is found within nucleotides 2 to 151 of SEQ ID NO: 1 or within nucleotides 32 to 170 of SEQ ID NO: 1. In various aspects of the methods disclosed herein, the step of amplifying the 171 base pair (bp) target DNA sequence, or fragment thereof, comprises the use of a forward primer selected from the group consisting of forward primers designed within nucleotides 2 to 151 of SEQ ID NO: 1 and a reverse primer designed within nucleotides 32 to 170 of SEQ ID NO: 1. In certain aspects, the forward primer is selected from the group consisting of forward primers encoded by SEQ ID NO: 2, SEQ ID NO: 3, and SEQ ID NO: 4 and the reverse primer is selected from the group consisting of reverse primers encoded by SEQ ID NO: 5, SEQ ID NO: 6, and SEQ ID NO: 7. In one aspect, the forward primer is encoded by SEQ ID NO: 4 and wherein said reverse primer is encoded by SEQ ID NO: 7.

In another aspect of the methods disclosed herein, the step of detecting the amplification products comprises using end-point assays, such as gel electrophoresis or sandwich assays, and/or detecting the amplification products in real time. In certain aspects, the step of detecting the amplification products comprises using fluorescence, and in certain aspects, the step of detecting the amplification products comprises using one or more probes. In certain aspects, the one or more probes are selected from the group consisting of fluorescent probes, non-fluorescent probes, and antigenically labeled probes, such as fluorescein- (e.g., FAM™-), digoxigenin-, and biotin-labeled probes, and in certain aspects, the one or more probes are nucleic acid probes. In certain aspects, the one or more probes are labeled with a reporter fluorophore at the 5′ end of the probe sequence and a quencher fluorophore at the 3′ end of the probe sequence, and in certain aspects, the reporter fluorophore is selected from the group consisting of fluoroscein, fluorescein isothiocyantate (FITC), 6-carboxy-2′,4,4′,5′,7,7′-hexachlorofluorescein, 6-carboxy-4′,5′-dichloro-2′,7′-dimethoxyfluorescein succinimidyl ester, and tetrachlorofluorescein, and the quencher fluorophore is selected from the group consisting of fluorescent (e.g., TAMRA) and non-fluorescent quenchers, such as dark quenchers, e.g., BLACK HOLE QUENCHER′ dyes including BHQ-1, BHQ-2, and BHQ-3 and dimethylaminoazobenzenesulfonic acid (Dabsyl).

In certain aspects, the step of detecting the amplification products comprises using immunochromatography, such as the use of a lateral flow immunoassay.

In another aspect of the methods disclosed herein, the sample is a human or animal sample, such as an equine sample.

In another aspect, the step of amplifying a 171-bp target DNA sequence, or fragment thereof, comprises the use of an isothermal amplification reaction, such as a recombinase polymerase amplification reaction.

In another aspect, the application relates to kits for detecting the presence of A. phagocytophilum in a sample according to the methods of the present application. The kits disclosed herein may comprise one or more forward primers selected from the group consisting of forward primers designed within nucleotides 2 to 151 of SEQ ID NO: 1 and one or more reverse primers designed within nucleotides 32 to 170 of SEQ ID NO: 1, wherein the kit may be used for detecting the presence of A. phagocytophilum in a sample and/or for diagnosing TBF in an animal and/or for diagnosing HGA in a human comprising using a nucleic acid amplification reaction. In certain aspects, the kit comprises one or more forward primers selected from the group consisting of forward primers encoded by SEQ ID NO: 2, SEQ ID NO: 3, and SEQ ID NO: 4 and one or more reverse primers selected from the group consisting of reverse primers encoded by SEQ ID NO: 5, SEQ ID NO: 6, and SEQ ID NO: 7, and in certain aspects, the forward primer is encoded by SEQ ID NO:4 and the reverse primer is encoded by SEQ ID NO: 7.

In another aspect, the kit further comprises one or more additional reagents for performing the nucleic acid amplification reaction, and in another aspect, the amplification reaction is an isothermal amplification reaction. In certain aspects, the one or more additional reagents are used for detecting the amplicon using endpoint assays and/or for detecting the amplicon in real time, and in another aspect, the one or more reagents are used for detecting the amplicon using agarose gel electrophoresis and/or using immunochromatography.

Another aspect of the present application relates to a method of treating or ameliorating a pathological condition caused by A. phagocytophilum in a subject in need thereof. The method comprises the steps of: (a) obtaining a biological sample from said subject; (b) detecting the presence of A. phagocytophilum in said biological sample from said subject by amplifying a 171-bp target DNA sequence encoded by SEQ ID NO: 1, or a fragment thereof, within the msp2 gene in the A. phagocytophilum genome and detecting the amplification products, wherein the detection of said amplification products indicates a positive diagnosis of said pathological condition caused by A. phagocytophilum in said subject; and (c) administering to said subject a therapeutically effective amount of an anti-A. phagocytophilum agent.

Also disclosed are methods of treating or ameliorating a pathological condition caused by A. phagocytophilum in a subject in need thereof, the method comprising the step of administering to said subject a therapeutically effective amount of an anti-A. phagocytophilum agent, wherein prior to the administering step the presence of A. phagocytophilum has been detected in a biological sample obtained from the subject by amplifying a 171-bp target DNA sequence encoded by SEQ ID NO: 1, or a fragment thereof, within the msp2 gene in the A. phagocytophilum genome and detecting the amplification products.

In certain aspects, of the method of treating or ameliorating a pathological condition caused by A. phagocytophilum, the subject is a human and the pathological condition is HGA, and in certain aspects, the subject is an animal, such as a horse, and the pathological condition is TBF. In certain aspects, a positive diagnosis is made within one week after infection by A. phagocytophilum. In certain aspects, the biological sample is a blood sample, and in certain aspects, the anti-A. phagocytophilum agent is an antibiotic, such as doxycycline.

### BRIEF DESCRIPTION OF THE DRAWINGS

The aspects and embodiments of the present application can be better understood by reference to the following drawings. The drawings are merely exemplary to illustrate certain features that may be used singularly or in combination with other features and the present application should not be limited to the embodiments shown.

FIGS. 1A, 1B, and 1C relate to the design and evaluation of RPA primers and probe for a conserved multicopy target DNA fragment in the A. phagocytophilum genome. FIG. 1A reflects the bioinformatics analysis based on the whole genome sequence of A. phagocytophilum HZ strain which identified a well-conserved multicopy DNA fragment located within msp2 (12 to 21 copies were found in various strains). FIG. 1B illustrates the three primers in either forward (F) or reverse (R) directions which were designed and tested using conventional PCR to amplify Anaplasma genomic DNA using different combinations of the primer sets as indicated. As depicted, the PCR products were analyzed by agarose gel electrophoresis. FIG. 1C depicts a schematic illustration of SEQ ID NO: 1 and the locations of forward and reverse primers and a fluorescent probe for an RPA assay of the present application. As provided therein, the arrow pointing to the right indicates the sequence of the RPA forward primer “AnaplasmaRPA 3F” of Table 1 (SEQ ID NO: 4) and the arrow pointing to the left indicates the sequence of the RPA reverse primer “AnaplasmaRPA 3R” of Table 1 (SEQ ID NO: 7). The dotted line indicates the sequence of the “Anaplasma fluorescent probe” of Table 1 (SEQ ID NO: 8). Reading from left to right, the first bolded “T” was tagged with FAM™; the bold “C” was replaced with THF; and the second bolded “T” was tagged with BHQ-1 as described herein.

FIGS. 2A-2B demonstrate the analytical limit of detection for the A. phagocytophilum RPA assay which is one genomic copy. FIG. 2A depicts the plasmid containing RPA target sequence which was diluted (1000 to 5 copies) and used as a template for RPA reactions. Data indicate that the RPA assay reliably detected the presence of 5 copies of plasmid within 10 minutes of reaction. Fluorescent signals were monitored in real time in a Twista® tube scanner. FIG. 2B depicts A. phagocytophilum (Webster strain) DNA of 1000 to 1 genomic copies which was used as a template for amplification by RPA. Data indicate that specific amplification was observed in reactions containing 1000 to as little as 1 genomic copy of A. phagocytophilum DNA.

FIGS. 3A-3B depict the high analytical specificity of the A. phagocytophilum RPA assay of the present application. FIG. 3A depicts genomic DNA from various organisms, including A. phagocytophilum (Webster strain, 5 genomic copies), Ehrlichia chaffeensis (E. chaffeensis, Liberty strain, 1×10⁴ copies), Borrelia burgdorferi (B31 strain, 1×10⁵ copies), Orientia tsutsugamushi (Karp strain, 2×10⁴ copies), Rickettsia rickettsia (2×10⁵ copies) and human (1×10⁵ copies), which were used as a template for RPA reactions. FIG. 3B depicts a summary of RPA results using DNA from various organisms (at least 2×10⁴ genomic copies from each organism were used except for A. phagocytophilum at 250 copies). Data in both FIG. 3A and FIG. 3B indicate that the assay specifically detected A. phagocytophilum DNA without amplification of excess genomic DNA from closely related bacteria.

FIGS. 4A-4C depict the high analytical and clinical sensitivity of the A. phagocytophilum RPA assay of the present application. FIG. 4A illustrates that DNA was extracted from 200 μL of normal human whole blood spiked with 0 to 250 copies of A. phagocytophilum genomic DNA and eluted into 20 μL elution buffer. Four microliters (⅕ of total elution) from each extraction was used for A. phagocytophilum RPA reactions. Summary of detection results using either real-time PCR (primer set msp2F/msp2R, Table 1) or RPA is shown (*, positive results out of total number of trials). FIG. 4B depicts representative real time fluorescent signals from RPA reactions using “expected copies per reaction” as in FIG. 4A. FIG. 4C depicts representative real time fluorescent signals from RPA reactions using 2 μL of DNA extracted from human patient blood samples (see also Table 2). Signals from an E. chaffeensis infection patient (99HE9) sample overlapped with normal human blood at the bottom of the graph. Experiments were repeated at least three times for each DNA sample.

FIG. 5 depicts the 171 bp sequence of the A. phagocytophilum msp2 gene which is a high copy DNA target of the assay of the present application (SEQ ID NO: 1).

### DETAILED DESCRIPTION

The following detailed description is presented to enable any person skilled in the art to make and use the subject of the application. For purposes of explanation, specific nomenclature is set forth to provide a thorough understanding of the present disclosure. However, it will be apparent to one skilled in the art that these specific details are not required to practice the subject of the application. Descriptions of specific applications are provided only as representative examples. The present application is not intended to be limited to the embodiments shown but is to be accorded the widest possible scope consistent with the principles and features disclosed herein.

Where applicable, all percentages and ratios used herein are by weight of the total composition unless otherwise indicated herein. All temperatures are in degrees Celsius unless specified otherwise. All measurements are made at 25° C. and normal pressure unless otherwise designated. Aspects and embodiments of the present disclosure can “comprise” (open ended) or “consist essentially of” the components of the present disclosure as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the aspect or embodiment may include elements in addition to those recited in the claim, but only if the additional elements do not materially alter the basic and novel characteristics of the claimed aspect or embodiment.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Unless otherwise indicated, as used herein, “a” and “an” include the plural, such that, e.g., “a sample” can mean at least one sample, as well as a plurality of samples, i.e., more than one sample.

As used herein, the term “and/or” when used in a list of two or more items means that any one of the listed characteristics can be present, or any combination of two or more of the listed characteristics can be present. For example, if an assay of the instant application is described as containing characteristics A, B, and/or C, the assay can contain A feature alone; B alone; C alone; A and B in combination; A and C in combination; B and C in combination; or A, B, and C in combination. The entire teachings of any patents, patent applications, or other publications referred to herein are incorporated by reference herein as if fully set forth herein.

As used herein, the terms “subject,” “a subject in need,” “a subject in need thereof,” “patient” and like terms may be used interchangeably and include an animal, including but not limited to birds and mammals, who may be infected by A. phagocytophilum and from whom a sample may be obtained for assay according to the methods of the present application. Human beings are also encompassed in these terms. In particular, subjects include, but are not limited to, domesticated animals as well as non-human primates and human patients.

In addition to humans, non-limiting examples of mammals include non-human primates (e.g., monkeys, chimpanzees), rodents (e.g., rats, mice, guinea pigs), lagomorphs, canines, felines, and livestock (e.g., bovine, porcine, equine, ovine, caprine).

In a first aspect, the application relates to a method of detecting the presence of A. phagocytophilum in a sample comprising (a) amplifying a 171-bp target DNA sequence encoded by SEQ ID NO: 1, or a fragment thereof, within the msp2 gene in the A. phagocytophilum genome; and (b) detecting the amplification products of step (a); wherein the detection of amplification products in step (b) indicates the presence of A. phagocytophilum in the sample.

In another aspect, the application relates to a method of diagnosing tick-borne fever (TBF) in an animal comprising detecting the presence of A. phagocytophilum in a sample from said animal, said method comprising a) amplifying a 171-bp target DNA sequence encoded by SEQ ID NO: 1, or a fragment thereof, within the msp2 gene in the A. phagocytophilum genome; and (b) detecting the amplification products of step (a); wherein the detection of said amplification products indicates a positive diagnosis of TBF in said animal.

In yet another aspect, the application relates to a method of diagnosing human granulocytic anaplasmosis (HGA) in a human comprising detecting the presence of A. phagocytophilum in a sample from said human, said method comprising a) amplifying a 171-bp target DNA sequence encoded by SEQ ID NO: 1, or a fragment thereof, within the msp2 gene in the A. phagocytophilum genome; and (b) detecting the amplification products of step (a); wherein the detection of said amplification products indicates a positive diagnosis of HGA in said human.

In some embodiments of the methods of the present application, the fragment of the 171-bp target DNA sequence is selected from the group consisting of nucleotide (nt) fragments at least about 50, 55, 60, 65, 70, 75, 80, 85, 90, 95, 100, 105, 110, 115, 120, 125, or 130 bp in length found within SEQ ID NO: 1. In some embodiments of the methods of the present application, the fragment of the 171-bp target DNA sequence is selected from the group consisting of nucleotide (nt) fragments at least about 100 bp in length found within SEQ ID NO: 1, such as about 100-110, 100-120, or 100-130 bp in length found within SEQ ID NO: 1. In certain embodiments, the fragment is any nucleotide fragment ranging from about 50 to about 100 bp long found within nt 2-nt 151 of SEQ ID NO: 1, or within nt 32-nt 170 of SEQ ID NO: 1. In certain embodiments, the fragment is any nucleotide fragment that is at least about 100 bp long found within nt 2-nt 151 of SEQ ID NO: 1, or within nt 32-nt 170 of SEQ ID NO: 1. In certain embodiments, the methods of the application comprise the use of a target nucleic acid sequence that has substantial identity to SEQ ID NO: 1 or a fragment thereof. In some embodiments the nucleic acid is DNA. In some embodiments, the nucleic acid is RNA.

In yet another aspect, the application relates to primers designed for use in the amplification step (a) of the methods of the present application. In a particular embodiment, the primers of the present application are employed in the methods of the present application as a combination or pair of a forward primer and a reverse primer. In various embodiments, the primers are designed based on the 171-bp target DNA sequence encoded by SEQ ID NO: 1, or a fragment thereof. In one embodiment, the forward primers comprise one or more forward primers designed based on nucleotides 2 to 151 of SEQ ID NO: 1. In another embodiment, the reverse primers comprise one or more reverse primers designed based on nucleotides 32 to 170 of SEQ ID NO: 1. In a particular embodiment, the primers comprise primers selected from the group consisting of primers encoded by SEQ ID NOs: 2-7. In a particular embodiment, the forward and reverse primer combination is selected from groups consisting of forward primers encoded by SEQ ID NO: 2, SEQ ID NO: 3, and SEQ ID NO: 4 and reverse primers encoded by SEQ ID NO: 5, SEQ ID NO: 6, and SEQ ID NO: 7. In various embodiments, the primer combination or pair includes any combination of SEQ ID NOs: 2-4 with SEQ ID NOs: 5-7. In particular embodiments, the primer pairs are SEQ ID NO: 2 and SEQ ID NO: 5; SEQ ID NO:2 and SEQ ID NO: 6; SEQ ID NO: 2 and SEQ ID NO: 7; SEQ ID NO: 3 and SEQ ID NO: 5; SEQ ID NO: 3 and SEQ ID NO: 6; SEQ ID NO:3 and SEQ ID NO: 7; SEQ ID NO: 4 and SEQ ID NO: 5; SEQ ID NO:4 and SEQ ID NO: 6; and SEQ ID NO: 4 and SEQ ID NO: 7. In a particular embodiment, a primer pair of the present application is the forward primer encoded by SEQ ID NO: 4 and the reverse primer encoded by SEQ ID NO: 7. In another embodiment, the primers are designed based on a nucleic acid sequence that has substantial identity to SEQ ID NO: 1 or a fragment thereof.

In another embodiment, the detecting amplification products step (b) of the methods of the present application comprises using end-point assays and/or detecting the amplification products in real time.

In a particular embodiment, said end-point assays comprise gel electrophoresis or sandwich assays.

In some embodiments, the detecting amplification products step (b) of the methods of the present application comprises using fluorescence.

In some embodiments, the detecting amplification products step (b) of the methods of the present application comprises using one or more probes. In particular embodiments, said probes are selected from the group consisting of fluorescent probes, non-fluorescent probes, and antigenically-labeled probes. In a particular embodiment, the probe is a nucleic acid probe. In another embodiment, the nucleic acid probe is encoded by SEQ ID NO: 8. In a particular embodiment, the antigenically-labeled probes are selected from the group consisting of fluorescein- (e.g., FAM™) digoxigenin- and biotin-labeled probes. In another particular embodiment, the probes are labeled with a reporter fluorophore at the 5′ end of the probe sequence and a corresponding and appropriate quencher fluorophore at the 3′ end of the probe sequence. In particular embodiments, the reporter fluorophores are selected from the group consisting of fluoroscein, fluorescein isothiocyantate (FITC), 6-carboxy-2′,4,4′,5′,7,7′-hexachlorofluorescein, 6-carboxy-4′,5′-dichloro-2′,7′-dimethoxyfluorescein succinimidyl ester, and tetrachlorofluorescein and the quencher fluorophores are selected from the group consisting of fluorescent (e.g., TAMRA) and non-fluorescent quenchers, such as dark quenchers, e.g., BLACK HOLE QUENCHER™ dyes including BHQ-1, BHQ-2, and BHQ-3 and dimethylaminoazobenzenesulfonic acid (Dabsyl).

In a particular embodiment, the detecting amplification products step (b) of the methods of the present application comprises using immunochromatography. In a particular embodiment, the immunochromatography comprises the use of a lateral flow immunoassay. In a particular embodiment, the lateral flow assay can detect biotin- or digoxigenin-labeled amplicons.

In various embodiments, the samples assayed according to the methods of the application are clinical samples. In particular embodiments, the samples are human or animal samples. In a particular embodiment, the animal is a domestic animal. In another particular embodiment, the domestic animal is an equine.

In particular embodiments, the amplification step (a) of the methods of the application comprises the use of an isothermal amplification reaction. In a particular embodiment, the isothermal amplification reaction is a recombinase polymerase amplification reaction (RPA).

In another aspect, the application relates to kits for detecting the presence of A. phagocytophilum in a sample according to the methods of the present application. Thus, in a particular embodiment, a kit of the application may comprise one or more forward primers selected from the group consisting of forward primers designed within nucleotides 2 to 151 of SEQ ID NO: 1, and one or more reverse primers designed within nucleotides 32 to 170 of SEQ ID NO: 1, wherein said kit may be used for detecting the presence of A. phagocytophilum in the sample and/or for diagnosing tick-borne fever (TBF) in an animal and/or for diagnosing human granulocytic anaplasmosis (HGA) in a human comprising using a nucleic acid amplification reaction. In a particular embodiment, the kit may comprise one or more forward primers selected from the group consisting of forward primers encoded by SEQ ID NO: 2, SEQ ID NO: 3, and SEQ ID NO: 4 and one or more reverse primers selected from the group consisting of reverse primers encoded by SEQ ID NO: 5, SEQ ID NO: 6, and SEQ ID NO: 7. In a particular embodiment, the forward primer is encoded by SEQ ID NO:4 and the reverse primer is encoded by SEQ ID NO: 7. In another embodiment, the kit may further comprise one or more additional reagents for performing the nucleic acid amplification reaction. In a particular embodiment, the nucleic acid amplification reaction is an isothermal amplification reaction. In a particular embodiment, the isothermal amplification reaction is a RPA. In various additional embodiments, the kits may further comprise one or more reagents for detecting an amplicon of the nucleic acid amplification reaction. In various particular embodiments, such one or more reagents for detecting an amplicon may be used for detecting an amplicon using endpoint assays and/or detecting the amplicon in real time. In various embodiments, the reagents provide for detecting the products of the amplification reaction using agarose gel electrophoresis and/or using immunochromatography.

Another aspect of the present application relates to a method of treating or ameliorating a pathological condition caused by A. phagocytophilum in a subject in need thereof. The method comprises the steps of: (a) obtaining a biological sample from said subject; (b) detecting the presence of A. phagocytophilum in said biological sample from said subject by amplifying a 171-bp target DNA sequence encoded by SEQ ID NO: 1, or a fragment thereof, within the msp2 gene in the A. phagocytophilum genome and detecting the amplification products, wherein the detection of said amplification products indicates a positive diagnosis of said pathological condition caused by A. phagocytophilum in said subject; and (c) administering to said subject a therapeutically effective amount of an anti-A. phagocytophilum agent.

Alternatively, the method of treating or ameliorating a pathological condition caused by A. phagocytophilum in a subject in need thereof, comprises the step of administering to said subject a therapeutically effective amount of an anti-A. phagocytophilum agent, wherein prior to the administering step the presence of A. phagocytophilum has been detected in a biological sample obtained from the subject by amplifying a 171-bp target DNA sequence encoded by SEQ ID NO: 1, or a fragment thereof, within the msp2 gene in the A. phagocytophilum genome and detecting the amplification products.

In some embodiments, the positive diagnosis is made within one week after infection by said A. phagocytophilum. In some embodiments, the biological sample is a blood sample. In some embodiments, the anti-A. phagocytophilum agent is an antibiotic. In some embodiments, the antibiotic is doxycycline.

In some embodiments, the subject is a human and said pathological condition is human granulocytic anaplasmosis (HGA).

In other embodiments, the subject is an animal and said pathological condition is tick-borne fever (TBF). In some further embodiments, the animal is a horse.

As discussed briefly above, the incidence of HGA has increased dramatically during the past 20 years and seroprevalences of 8.9 to 36% have been reported in certain parts of the United States (Aguero-Rosenfeld et al. 2002 J Clin Microbiol 40:2612-2615; Bakken et al. 1998 Clin Infect Dis 27:1491-1496). Although the case fatality rate is low at 0.6%, 36% of the patients develop disease severe enough to require hospitalization (Dahlgren et al. 2011 Am J Trop Med Hyg 85:124-131). Compared with traditional diagnostic methods, such as blood smear microscopy, serology and culture, direct pathogen DNA detection offers sensitive and rapid diagnosis during the early acute phase of the infection, which is critical for effective antibiotic treatment. However, PCR-based assays (Silaghi et al. 2017 Vector Borne Zoonotic Dis 17:12-22) require expensive equipment and trained operators, which are not available in rural areas where the infection is more likely to occur. Simple, rapid and low-cost methods are in urgent need in these areas. In particular, there remains the need for point-of-care diagnostic methods and tools, as well as methods for vector surveillance and epidemiologic studies, particularly in resource-constrained regions where other A. phagocytophilum detection methods are not readily available.

Accordingly, in view of existing needs, the present application relates to methods and materials for detecting A. phagocytophilum based on targeting a highly conserved multicopy 171 bp genomic region, or a fragment thereof, in the msp2 gene A. phagocytophilum. In a particular embodiment, the present application relates to a rapid, highly sensitive, and specific isothermal RPA assay for detecting A. phagocytophilum based on amplifying this highly conserved multicopy genomic region in A. phagocytophilum, or a fragment thereof. As provided in the below examples, data show that an assay of the present application has a limit of detection of one genomic copy of A. phagocytophilum DNA within 10 minutes of reaction, and displays 100% sensitivity and specificity in limited number of clinical samples. Accordingly, it is contemplated herein that an assay of the present application may be employed as a point-of-care diagnostic tool, and also used in methods for vector surveillance and epidemiologic studies, particularly in resource-constrained regions where other A. phagocytophilum point-of-care diagnostic tools and/or detection methods are not readily available. Specifically, in this regard, it is contemplated herein that the improved methods of detecting A. phagocytophilum in a sample disclosed herein also permit enhanced clinical use, e.g., improved methods for diagnosing tick-borne fever and/or human granulocytic anaplasmosis in subjects in need thereof.

As described herein, a method of the present application comprises detecting A. phagocytophilum in a sample by targeting the 171 bp sequence of the msp2 gene provided herein as SEQ ID NO: 1, or a fragment thereof. It is also understood herein that in various embodiments, the target nucleic acid can be in the context of genomic DNA, as well as RNA, amplification products, or other extraneous material.

It is understood herein that the target sequence for amplification in a method of the application includes SEQ ID NO: 1 in its entirety as well as fragments thereof. As contemplated herein, “a fragment thereof” is a portion of the 171 bp sequence encoded by SEQ ID NO: 1 which is sufficient in length to permit detection of A. phagocytophilum. As one of skill in the art will appreciate, the length of a target fragment of the present application may depend on the primers and probes used in the amplification methods. Thus, in a particular embodiment, it is contemplated herein that useful target fragments of the 171 bp sequence may be any fragment from within the 171 bp sequence that ranges from about 50 to about 100 bp long. In various embodiments, the fragment is any fragment about 100 bp long within nt2 and nt170 of the 171 bp sequence.

It is further contemplated herein that the definition of target sequences of the application can also include nucleic acid sequences that have substantial identity to SEQ ID NO: 1 or fragments thereof. As understood herein, “substantial identity” refers to a sequence of a percent identity that permits the specific detection of A. phagocytophilum from a sample. One of skill in the art will appreciate that target sequences of “substantial identity” to SEQ ID NO: 1 or fragments thereof may include not only target sequences comprising less than the entire 171 bp sequence, but also sequences comprising the entire 171 bp sequence as well as additional base pairs, e.g., one or more additional base pairs found before the beginning of the 171 bp sequence and/or one or more additional base pairs found after the end of the 171 bp sequence.

One of skill in the art may identify such sequences using conventional methods and without undue experimentation. In particular embodiments, it is contemplated herein that target sequences having substantial identity to SEQ ID NO: 1 or fragments thereof may be a sequence that has at least 50% sequence identity to the reference sequence. In additional embodiments, the percent identity may be any integer from 50% to 100%, including but not limited to 50%, 55%, 60%, 65%, 70%, 75%, 80%, 85%, 90%, 95%, or 99% sequence identity.

Also included in the present application are targets encoded as RNA based on the DNA sequence encoded by SEQ ID NO: 1 or fragments thereof, as well as based on nucleic acid sequences that have substantial identity to SEQ ID NO: 1 or fragments thereof as described herein.

As used herein, the terms “amplicon,” “amplified product,” “amplification product” and like terms include but are not limited to any polynucleotide generated as a copy of an original target sequence and/or a complementary sequence of target nucleotide sequence. The amplified target nucleic acid can be RNA or DNA or a modification thereof. If the target nucleic acid is RNA, the RNA can be directly amplified or can first be reversed transcribed into cDNA using a reverse transcriptase primer and reverse transcriptase according to conventional methods. Indeed, the use of RNA as the target is also a possible alternative if an RT-RPA, or a RT-LAMP or RT-qPCR method is applied according to the methods of the present application.

The term “primer” or “primers” used herein are familiar to one of skill in the art and refer to any oligonucleotide sequence that can hybridize or bind to a target nucleic acid sequence and be used to provide a starting point for DNA synthesis in a nucleic acid amplification reaction, including but not limited to, a PCR or an isothermal amplification reaction. Similarly, the terms “forward primer” and “reverse primer” are familiar to one of skill in the art, and refer to the design of primers complementary to nucleic acid sequences “upstream” (forward) and “downstream” (reverse) of a target sequence. In particular embodiments, with regard to the present application, it is contemplated herein that various multiple forward primers can be designed from nucleotides 2 to 151 of the target 171 bp sequence, and various multiple reverse primers can be designed within nucleotides 32 to 170 of the target 171 bp sequence provided herein as SEQ ID NO: 1. One of skill in the art may create such primer sequences using conventional methods and without undue experimentation. Examples of primers utilized by the methods of the present application include the forward and reverse primers listed in Table 1 herein as SEQ ID NOs: 2-7.

Additional materials and reagents for performing the nucleic acid amplification reaction of the methods of the present application are familiar to one of skill in the art and may be obtained from a variety of commercial vendors. As discussed in more detail below, these materials and reagents include, e.g., buffers, salts, metals, ions, unincorporated nucleotides, excess labels, and proteins. In particular embodiments, enzymes for use in the methods of the present application include, e.g., bacterial DNA recombinases, DNA binding proteins, and DNA polymerases familiar to one of skill in the art.

Similarly, materials and methods for detecting the amplification products of nucleic acid amplification reactions according to the disclosed methods are familiar to one of skill in the art and are discussed in detail below. In a particular embodiment, it is contemplated herein that the amplification products may be detected in “real time,” i.e., as the products are generated in the reaction. In another embodiment, it is also contemplated herein that amplicons may be detected at the end of the amplification reaction, e.g., using “end-point” assays familiar to one of skill in the art.

It is contemplated herein that various types of samples may be assayed for the presence of A. phagocytophilum according to the methods of the present application. Such samples include anything from which DNA or other types of nucleic acid may be obtained, including, for example, swabs of objects or other surfaces in the environment. In addition, in a particular embodiment, samples for assay according to the methods of the disclosure are biological samples. As used herein, the term “biological sample” includes any sample obtained from a living organism, including but not limited to animals, e.g., mammals, or other organisms. In particular embodiments, the samples are obtained from humans, and/or domestic or wild animals. In a particular embodiment, samples are obtained from equines, e.g., horses. In particular embodiments, the samples may be clinical samples.

### Amplification Reactions

It is contemplated herein that the target sequences disclosed herein may be detected using a variety of nucleic acid amplification methods familiar to one of skill in the art. These methods include, e.g., the polymerase chain reaction (PCR). See, e.g., McPherson et al, editors, PCR: A Practical Approach and PCR2: A Practical Approach IRL Press, Oxford, 1991 and 1995. PCR methodologies include, e.g., reverse transcription PCR, real-time PCR, nested PCR, quantitative PCR and multiplexed PCR. For example, Table 1 discloses primer sequences for qPCR that were used in the examples provided below, e.g., msp2F and msp2R and ApMSP2f and ApMSP2r.

In addition to PCR, in a particular embodiment, the methods of the application comprise the use of isothermal recombinase polymerase amplification (RPA) assays to amplify the 171 bp sequence (SEQ ID NO: 1) or fragments thereof and allow detection of A. phagocytophilum according to the methods of the application. As appreciated by one of skill in the art, RPA assays are nucleic acid amplification methods that utilize DNA polymerase, but use a recombinase-primer complex which permits the isothermal amplification of nucleic acid without using thermal cycling procedures required in conventional PCR. One of skill in the art will appreciate that RPA assays include, e.g., nucleic acid sequence-based amplification, strand displacement amplification, and multiple displacement amplification. See, e.g., Fakruddin et al., J. Pharm Bioallied Sci. 2013 October-December; 5(4):245-252; Li et al., Analyst, 144:31-67 (2019), the entire contents of which are incorporated by reference herein.

RPA assays include isothermal amplification reactions such as ramification amplification methods (Zhang et al., Molecular Diagnosis, 6(2):141-150 (2001)); helicase-dependent amplification (Vincent, et al., EMBO Reports, 5(8):795-800 (2004)); rolling circle amplification (RCA) (Gu et al., Pharmaceuticals 2018, 11:35; Zhao, et al., Angew. Chem. Int. Ed. 2008, 47, 6330-6337); and loop-mediated isothermal amplification (LAMP) (Notomi et al., Nucleic Acids Res 28:E63 (2000). One or more of such nucleic acid amplification methods may be performed by one of skill of the art using conventional methods, employing the target nucleic acid sequences and primer sequences disclosed herein, to practice the methods of the present application.

In a particular embodiment, it is contemplated herein that the methods of the present application may be practiced by performing an isothermal amplification reaction in method step (a) such as provided in Piepenburg, et al., PLOS Biology, 4(7):1115-1121 (2006). As particularly provided therein, the isothermal amplification reaction comprises the use of liquid or lyophilized enzymes such as DNA recombinase (e.g., uvsX and uvxY); single-stranded DNA binding protein (e.g., gp32) and DNA polymerase (e.g., Bsu). The specific reaction reagents and conditions described therein also include the use of 50 mM Tris (pH 7.9), 100 mM potassium acetate, 14 mM magnesium acetate, 2 mM DTT, 5% Carbowax™ (20M), 200 μM dNTPs, 3 mM ATP, 50 mM phosphocreatine, 100 ng/μl creatine kinase, 30 ng/μl Bsu, 900 ng/μl gp32, 120 ng/μl uxsX, and 30 ng/μl uvsY, rehydration buffer (Poly(oxy-1,2-ethanediyl) hydro-ω-hydroxy-Ethane-α-1,2-diol, ethoxylated, Potassium acetate in Tris), magnesium acetate, and deoxynucleotides, along with the proper concentration of primers and probe. As provided therein, these reagents are mixed with the appropriate DNA template and incubated at 37 to 39° C. for 5 to 30 minutes and followed by appropriate methods of detection of amplifications. Of course, the specific isothermal amplification reaction conditions disclosed in Piepenburg and provided above is only one example; it is understood herein that such methods may be modified in one or more aspects by one of skill in the art without undue experimentation for use in the methods of the present application.

It is further contemplated herein that RNA sequences, as well as DNA sequences encoding the target 171 bp sequence (or fragments thereof), may be employed in the methods of the present application. Indeed, in a particular embodiment, the use of RNA as the target nucleic acid is also a possible alternative, particularly if an RT-RPA, or an RT-LAMP or RT-qPCR method is applied. Such alternative methods may be performed by one of skill in the art using conventional methods.

As one of skill in the art will appreciate, laboratory equipment, reagents, enzymes and other proteins, etc. necessary for performing nucleic acid amplification reactions such as PCR and isothermal amplification reactions for use in the methods of the present application are familiar to one of skill in the art and may be obtained from a variety of commercial vendors, including but not limited to: Promega Corp (Madison, Wis.); QIAGEN® (Hilden, Germany); Thermo Fisher Scientific (Waltham, Mass.), Abbott Laboratories (Chicago, Ill.) and TwistDx™ (Cambridge, UK).

### Methods of Detecting Amplification Products

It is contemplated herein that the amplification products created in step (a) may be detected in step (b) of the disclosed methods using a variety of conventional methods familiar to one of skill in the art. Various qualitative and/or quantitative nucleic acid assays, as well as instrument-based and non-instrument-based detection methods, are contemplated herein. In particular embodiments, the methods of the present application may comprise end-point detection of amplification products as well as real-time detection methods. Since real-time detection methods may be employed, it is contemplated herein that the amplification step (a) and detection step (b) of the disclosed methods may be performed concurrently.

Accordingly, as one of skill in the art will appreciate, techniques for amplicon detection for use in the methods of the present application include, but are not limited to, polyacrylamide or agarose gel electrophoresis; high resolution capillary electrophoresis; real-time fluorescence (probe- or binding dye-based); liquid-phase bead arrays, 2D solid-phase printed microarrays (endpoint array hybridization), or hybridizing probe-based luminescence detection. To this end, one of skill in the art will also appreciate that amplicon detection methods for use with the present application may comprise using one or more various tags, probes, and labels. These include, e.g., hybridizing probes including unlabeled probes or labeled probes, e.g., probes comprising fluorescent labels, antigenic labels, and/or radioactive labels; fluorescent dyes or fluorophores; and/or DNA intercalating agents. Various methods may comprise using tagged forward and reverse primers, e.g., end-point “sandwich assays” using antigenic labels such as fluorescein/anti-fluorescein antibodies, biotin/streptavidin, or biotin, digoxigenin, and FAM™, for capture and detection. See, e.g., materials and methodologies available from commercial vendors such as Milenia Biotech GmbH (Giessen, Germany) or TwistDx™ (Cambridge, UK). Additional labelling techniques may include the use of silver nitrate, ethidium bromide, and/or biotin/avidin-horseradish peroxidase according to conventional methods.

Additional conventional methods for detecting amplicons familiar to one of skill in the art include methods comprising the real time detection of turbidity derived from magnesium pyrophosphate formation (Mori et al., BBRC, 2001 Nov. 23; 289(1):150-154). Additional amplicon detection methods include the use of antibodies and immunoassays such as lateral flow “dipstick” immunoassay systems, including commercially available rapid strip tests. See, e.g., reagents and methodologies available from Abingdon Health (York, UK). It is contemplated herein that all the above-indicated methods may be performed by one of skill in the art using conventional methods and reagents obtained from commercial vendors.

In various embodiments, antigenically-labeled probes for use in the methods of the present application include fluorescein-labeled (e.g., FAM™-labeled), digoxigenin-, or biotin-labeled probes. In a particular embodiment, the probes can be used with ready-to-use commercially available lateral flow immunochromatographic rapid tests to detect fluorescein-labeled (e.g., FAM™-labeled), biotin-labeled or digoxigenin-labeled amplicons.

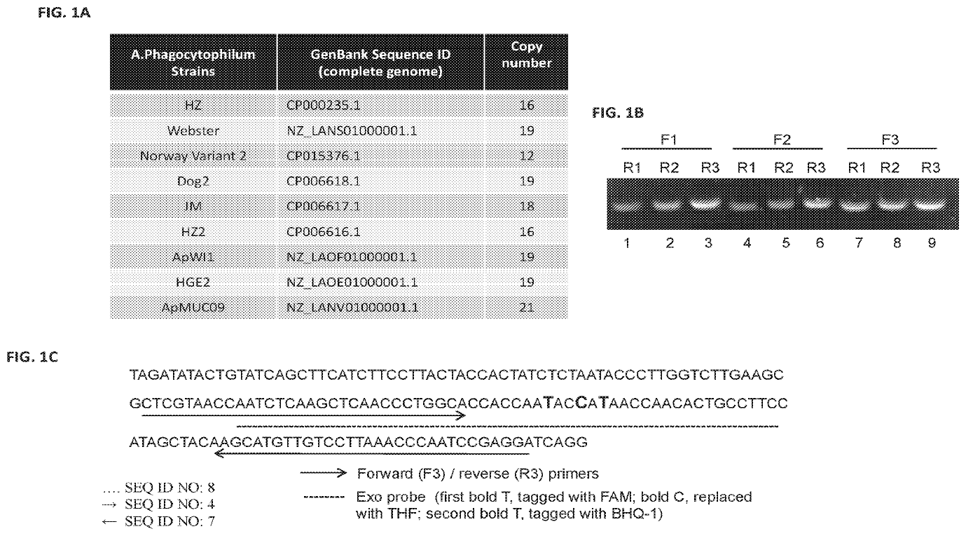

In another particular embodiment, probes for use in the methods of the present application include probes labeled at the 5′ end with a fluorescent reporter dye and probes labeled at the 3′end with an appropriate corresponding quencher dye. Suitable combinations of reporter fluorophores and quencher dyes are known and other combinations may be determined by one of skill in the art without requiring undue experimentation. Possible conventional reporter dyes include but are not limited to xanthene and amine-reactive dyes, e.g., fluorescein and derivatives thereof. Various fluorophores include, e.g., fluorescein (FAM™), fluorescein isothiocyantate (FITC), 6-carboxy-2′,4,4′,5′,7,7′-hexachlorofluorescein (HEX™), 6-carboxy-4′,5′-dichloro-2′,7′-dimethoxyfluorescein, succinimidyl ester (JOE™ dyes) and tetrachlorofluorescein (TET′). Corresponding and appropriate quencher dyes which may be employed with reporter probes such as FAM™, FITC, HEX™, Joe™ or TET™ are familiar to one of skill in the art and include fluorescent or non-fluorescent quenchers, e.g., dark quenchers such as BLACK HOLE QUENCHER dyes BHQ-1, BHQ-2, and BHQ-3 as well as dimethylaminoazobenzenesulfonic acid (Dabsyl), and TAMRA. (See, e.g., FIG. 1). Reporter and quencher fluorophores, and methods of use thereof, are familiar to one of skill in the art and are available from commercial vendors. See, e.g., reagents and methodologies available from MilliporeSigma (St. Louis, Mo.); Biolegio (Nijmegen, The Netherlands); Integrated DNA Technologies (Coralville, Iowa); Biosearch Technologies (Petaluma, Calif.); and Eurofins MWG (Huntsville, Ala.).

One of skill in the art will appreciate that the choice of the probes for use with the methods of the present application will depend upon the detector that is used for amplicon detection. In a particular embodiment, a detector that is capable of detecting fluorescent signals from any of the aforementioned reporter fluorophores can be used for detection. For example, in one possible embodiment, the detecting amplification products step (b) of the methods of the present application comprises using immunochromatography. In a particular embodiment, the immunochromatography comprises the use of a lateral flow immunoassay that can detect biotin or digoxigenin labeled amplicons. The detection of the presence of biotin labeled or digoxigenin labeled probes can be performed using methods familiar to one of skill in the art and using commercially available reagents and systems. See, e.g., reagents and methodologies available as HybriDetect 1 (Milenia Biotec, Giessen, Germany) or the U-Star Disposable Nucleic Acid Lateral Flow Detection Units (TwistDx™, Cambridge, UK).

It is contemplated herein that the primers and target nucleic acids disclosed herein may be used with amplification and detection methods to produce rapid, sensitive, and reliable target amplification and amplicon detection while minimizing the production of artifacts and false negatives. In various embodiments, such methods may also include the use of appropriate internal controls familiar to one of skill in the art. See, e.g., Niemz et al. Trends Biotechnol. 2011 May; 29(5):240-250. Accordingly, it is contemplated herein that the methods of the present application may be used for point-of-care diagnostics.

In a particular embodiment, it is contemplated herein that the primers and target nucleic acid disclosed herein to detect A. phagocytophilum may be used with an isothermal amplification reaction. In a particular embodiment, the isothermal amplification reaction is recombinase polymerase amplification (RPA). In another particular embodiment, the steps of the disclosed method comprise the use of an RPA assay in combination with a nucleic acid lateral flow immunoassay detection method.

As described in the below examples, in a particular embodiment of the application the amplification product is produced using RPA according to, or in a manner similar to that disclosed in Piepenburg et al., PLoS Biol, 2006 July: 4(7) e204, the contents of which are incorporated by reference herein. It is further contemplated that the amplicons may be detected using a fluorescent nucleic acid probe using commercially available methods and reagents, e.g., such as those available from TwistDx™ (Cambridge, United Kingdom). In this regard, a fluorescent nucleic acid probe is depicted in FIG. 1 and contains FAM™, THF, and BHQ. As depicted therein, and according to this commercially available detection system, THF (tetrahydrofuran) is used to replace “regular DNA bases” (in this case, it is a C (cytosine) in the sequence that is replaced) which results in a “space in the DNA sequence” that can be recognized by an exonuclease. The exonuclease cuts and replaces the rest of the oligonucleotides with the correct oligonucleotides and thus replaces the BHQ-labeled half of the nucleotides to allow FAM™ signals to be detectable.

In this particular commercially available detection method, a tube is used as both an incubator and a detector. To amplify extremely low copy template, four minutes after the start of reaction, samples are quickly removed from the tube scanner and vortexed to mix one more time before incubation at 39° C. for another 16 minutes. The reaction time can be in the range of 5 minutes to 1 hour for positive detection of amplification products. The fluorescent signal can be monitored for as long as the reaction time or it can be started after the short vortexing four minutes after the start of reaction. Fluorescence signal is monitored and analyzed using the Twista® Studio software (TwistDx™, Cambridge, United Kingdom).

While commercially available detection systems may be used out of convenience, it is understood herein that one of skill in the art may also obtain all the necessary reagents for use in the methods of the present application from one or more commercial vendors.

### Kits

The present application also relates to kits for performing the methods of the present application. Thus, in various embodiments, the kits may contain one or more reagents for detecting the presence of A. phagocytophilum in a sample and/or for diagnosing TBF in an animal and/or for diagnosing HGA in a human according to the methods of the application. In a particular embodiment, it is contemplated herein that such kit may comprise one or more forward primers selected from the group consisting of forward primers designed within nucleotides 2 to 151 of SEQ ID NO: 1, and one or more reverse primers designed within nucleotides 32 to 170 of SEQ ID NO: 1. In a particular embodiment, the one or more forward primers is selected from the group consisting of forward primers encoded by SEQ ID NO: 2, SEQ ID NO: 3, and SEQ ID NO: 4 and the one or more reverse primers is selected from the group consisting of reverse primers encoded by SEQ ID NO: 5, SEQ ID NO: 6, and SEQ ID NO: 7. In a particular embodiment, the kit comprises the primer pair encoded by SEQ ID NO:4 and SEQ ID NO: 7.

In various additional embodiments, the kit may further comprise one or more additional reagents for nucleic acid amplification. In a particular embodiment, the nucleic acid amplification is an isothermal amplification reaction, including but not limited to an RPA reaction. The kit may further comprise one or more reagents for detecting an amplicon, including but not limited to probes and tags for labeling and detecting nucleic acids according to conventional methods such as disclosed in detail hereinabove, including but not limited to detecting amplicons using endpoint assays and/or detecting the amplification products in real time, including but not limited to detecting amplicons using agarose gel electrophoresis and/or using immunochromatography.

### Treatment

Clinical diagnosis of HGA and TBF is challenging as many subjects present with nonspecific symptoms and signs. Additionally, traditional diagnostic methods, such as cell counting in blood smears, can be time-consuming and error prone. The methods of the present disclosure provide earlier, more accurate detection or diagnosis of HGA or TBF in a subject, allowing earlier treatment of a subject in need thereof. Antibiotic treatment is most effective during the early course of the infection. Thus, it is contemplated herein that the present application also relates to a method of treating a pathological condition caused by A. phagocytophilum in a subject in need thereof comprising administering to said subject a therapeutically effective amount of an anti-A. phagocytophilum agent, wherein the subject has been identified as being in need of treatment using the A. phagocytophilum detection methods described herein. Thus, in certain embodiments, prior to treatment, a biological sample was obtained from the subject and tested for the presence of A. phagocytophilum by amplifying a 171-bp target DNA sequence encoded by SEQ ID NO:1, or a fragment thereof, within the msp2 gene in the A. phagocytophilum genome and detecting the amplification products, wherein the detection of said amplification products indicates a positive diagnosis of said pathological condition caused by A. phagocytophilum in said subject. Alternatively the method of treating a pathological condition caused by A. phagocytophilum in a subject in need thereof comprises (a) obtaining a biological sample from said subject; (b) detecting the presence of A. phagocytophilum in said biological sample from said subject by amplifying a 171-bp target DNA sequence encoded by SEQ ID NO:1, or a fragment thereof, within the msp2 gene in the A. phagocytophilum genome and detecting the amplification products, wherein the detection of said amplification products indicates a positive diagnosis of said pathological condition caused by A. phagocytophilum in said subject; and (c) administering to said subject a therapeutically effective amount of an anti-A. phagocytophilum agent. In various embodiments, the pathological condition is HGA or TBF. In other embodiments, the positive diagnosis is made within one week after infection by said A. phagocytophilum. In some embodiments, the biological sample is a blood sample. In additional embodiments, the anti-A. phagocytophilum agent is an antibiotic.

HGA in humans is treatable with broad spectrum antibiotics. In some embodiments, an adult subject, or a child subject who is at least 8 years of age, in need of treatment for HGA is treated with a therapeutically effective amount of at least one antibiotic that is a tetracycline class antibiotic. In some further embodiments, the tetracycline class antibiotic is selected from the group consisting of tetracycline, doxycycline, lymecycline, minocycline, sarecycline, oxytetracycline, and chlortetracycline. In particular embodiments, the subject is treated with a therapeutically effective amount of doxycycline. In some embodiments, a tetracycline class antibiotic can be co-administered with, or replaced by, a non-tetracycline class antibiotic including, but not limited to, penicillin, erythromycin, amoxicillin, cefuroxime, or azithromycin.

In some embodiments, an adult subject in need of treatment for HGA is treated with about 100, 150, 200, 250 or 300 mg of doxycycline/day. In particular embodiments, an adult subject is treated with about 200 mg of doxycycline/day. In some embodiments, the daily dosage is divided into two equal doses/day.

In some embodiments, a child subject in need of treatment for HGA who is at least 8 years of age is treated with about 3.5, 4.0, 4.4, or 5.0 mg/kg of doxycycline/day, up to an adult dosage. In a particular embodiment, a child subject who is at least 8 years of age is treated with about 4.4 mg/kg of doxycycline/day, up to an adult dosage. In some embodiments, the daily dosage is divided into two equal doses/day.

In some embodiments, a child subject in need of treatment for HGA who is less than 8 years of age, or any subject who is sensitive/allergic to tetracycline class antibiotics, can be treated with a therapeutically effective amount of amoxicillin or cefuroxime.

In some embodiments, a child subject in need of treatment for HGA is treated with a dosage of amoxicillin equivalent to about 25, 50 or 75 mg/kg three times/day, up to a maximum of 500 mg/dose. In a preferred embodiment, a child subject is treated with a dosage of amoxicillin equivalent to about 50 mg/kg three times/day, up to a maximum of 500 mg/dose.

In some embodiments, an adult subject in need of treatment for HGA is treated with a dosage of amoxicillin equivalent to about 125, 200, 250, 400, 500, 600, 775, or 875 mg/kg three times/day. In a preferred embodiment, an adult subject is treated with a dosage of amoxicillin equivalent to about 500 mg/dose three times/day.

In some embodiments, a child subject in need of treatment for HGA is treated with a dosage of cefuroxime equivalent to about 25, 30 or 35 mg/kg two times/day, up to a maximum of 500 mg/dose. In a preferred embodiment, a child subject is treated with a dosage of amoxicillin equivalent to about 30 mg/kg two times/day, up to a maximum of 500 mg/dose.

In some embodiments, an adult subject in need of treatment for HGA is treated with a dosage of cefuroxime equivalent to about 250, 400, 500, 600, or 750 mg/kg two times/day. In a preferred embodiment, an adult subject is treated with a dosage of cefuroxime equivalent to about 500 mg/dose two times/day.

TBF in non-human animals is treatable with broad spectrum antibiotics. In a particular embodiment, the non-human animal is a horse. In some embodiments, an animal subject in need of treatment for TBF is treated with a therapeutically effective amount of at least one antibiotic that is a tetracycline class antibiotic. In some further embodiments, the tetracycline class antibiotic is selected from the group consisting of tetracycline, doxycycline, lymecycline, minocycline, sarecycline, oxytetracycline, and chlortetracycline. In particular embodiments, the animal subject is treated with a therapeutically effective amount of doxycycline. In some embodiments, a tetracycline class antibiotic can be co-administered with, or replaced by, a non-tetracycline class antibiotic including, but not limited to, penicillin, erythromycin, amoxicillin, cefuroxime, or azithromycin.

In some embodiments of the present methods, a course of treatment lasts for about 10, 14, 21, 28, or 35 days. It is also envisioned in the present disclosure that, in some subjects/situations, a practitioner may prescribe a course of treatment that is longer, shorter, or intermediate to the previously mentioned time courses.

In some embodiments, an antibiotic of the present methods can be administered orally including, but not limited to, as a tablet, suspension, dragée, capsule, caplet, enteric-coated tablet/caplet, syrup, elixir, spirit or chewable. In some embodiments, an antibiotic of the present methods is a suppository. In some embodiments, an antibiotic of the present methods is administered as an injection including, but not limited to, intravenously, intramuscularly, or subcutaneously.

Although the application herein has been described with reference to particular embodiments, it is to be understood that these embodiments, and examples provided herein, are merely illustrative of the principles and applications of the present application. It is therefore to be understood that numerous modifications can be made to the illustrative embodiments and examples, and that other arrangements can be devised without departing from the spirit and scope of the present application as defined by the appended claims. All patent applications, patents, literature, and references cited herein are hereby incorporated by reference in their entirety.

### EXAMPLES

The following materials and methods were used to perform the experiments outlined in the below examples.

Sequence Analysis of A. phagocytophilum:

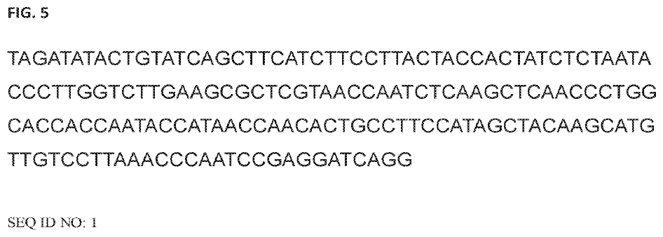

The whole genome sequence (bases 1 to 1,471,282) of A. phagocytophilum (HZ strain) was downloaded from the NCBI database (accession number: NC 007797.1). See also GenBank: CP000235.1. A 171-bp DNA fragment within the msp2 gene was found to have 16 copies using a sequence analysis software developed by Aplix Research Inc. (North Potomac, Md.). The 171 bp DNA fragment is provided in FIG. 5 as SEQ ID NO: 1.

### Primer and Probe Design:

Forward and reverse primers for RPA assay were designed using primer3 software (version 0.4.0) (Untergasser et al., 2012 Nucleic Acids Res 40:e115) and manually extended in the 5′ direction to 30 base pair in length. Primers for real-time PCR were designed based on the same 171-bp region using the online “Assay Design Center” (Roche Diagnostics, Corp. Indianapolis, Ind.). All primers were synthesized by Eurofins Genomics (Louisville, Ky.). Fluorescence-labeled probe was designed according to the manual from TwistDx™ (Cambridge, United Kingdom) and synthesized by LGC Biosearch™ Technologies (Petaluma, Calif.). Exemplary primer/probe sequences used in this application are listed in Table 1.

TABLE 1
RPA and qPCR primers (5 prime to 3 prime direction)

| | | SEQ |
|---|---|---|
| Primer Name | Primer Sequence | NO: |
| AnaplasmaRPA_1F | TCTAATACCCTTGGTCTTGAAGCGCTCGTA | 2 |
| AnaplasmaRPA_2F | TGGTCTTGAAGCGCTCGTAACCAATCTCA | 3 |
| AnaplasmaRPA_3F | CTCGTAACCAATCTCAAGCTCAACCCTGG | 4 |
| AnaplasmaRPA_1R | CATGCTTGTAGCTATGGAAGGCAGTGTTG | 5 |
| AnaplasmaRPA_2R | CTGATCCTCGGATTGGGTTTAAGGACAAC | 6 |
| AnaplasmaRPA_3R | TCCTCGGATTGGGTTTAAGGACAACATGC | 7 |
| Anaplasma fluorescent | AATCTCAAGCTCAACCCTGGCACCACCAA | 8 |
| probe | (FAM)]AC[dSpacer]A[T(BHQ- | |
| | 1)]AACCAACACTGCCTTC-[SpacerC3] | |
| msp2F | GTCTTGAAGCGCTCGTAACC | 9 |
| msp2R | GCTTGTAGCTATGGAAGGCAGT | 10 |
| Reference plasmid primer | CAGTCGTGAATGTAGAGGGAAAAAC | 11 |
| ankA-F | Dong et al. 2013 PLoS ONE 8:e74796 | |
| Reference plasmid primer | GGAATCCCCCTTCAGGAACTTG | 12 |
| ankA-R | Dong et al. 2013 | |
| ApMSP2f | ATGGAAGGTAGTGTTGGTTATGGTATT | 13 |
| | Courtney et al. 2004 | |
| ApMSP2r | TTGGTCTTGAAGCGCTCGTA | 14 |
| | Courtney et al. 2004 | |

### DNA Preparation and Quantification:

E. chaffeensis (Liberty strain) DNA was provided by BEI Resources (Manassas, Va.). A. phagocytophilum (Webster strain) was grown in human HL-60 cells. The culture was harvested and stored in liquid nitrogen when the number of bacteria reached about 50-100 bacteria per cell. After thawing, DNA extraction was performed using a QIAGEN® DNA mini kit (Germantown, Md.) following manufacturer's protocol for Gram-negative bacteria. DNA absorbance was measured on a Nanodrop™ 2000 spectrophotometer. Genomic copy numbers of A. phagocytophilum were quantified by a standard curve generated from serial dilution of a reference plasmid containing an ankA gene fragment on a 7500 Fast Real-Time PCR System (Applied Biosystems®, Foster City, Calif.). DNA from human whole blood was extracted using a QIAGEN® DNA mini kit following manufacturer's protocol for whole blood.

### PCR, Cloning and Real-Time PCR:

PCR was performed using Platinum® PCR SuperMix High Fidelity from Thermo Fisher Scientific (Waltham, Mass.) according to manufacturer's instructions. Initial evaluation of RPA primer sets was carried out in a PCR thermal cycler for 18 cycles (95° C., 20 seconds; 64° C., 20 seconds and 68° C., 40 seconds) followed by agarose gel electrophoresis. DNA fragments for ankA (primer set ankA-F/ankA-R, Table 1) and msp2 (primer set AnaplasmaRPA 1F/AnaplasmaRPA 2R, Table 1) were amplified for 18 and 16 cycles, respectively, followed by PCR amplicon purification immediately and TOPO® cloning into pCR-XL-TOPO vector (Thermo Fisher Scientific, Waltham, Mass.). Quantitative real-time PCR using QuantiFast SYBR® Green PCR kit (QIAGEN®, Germantown, Md.) was performed on a 7500 Fast Real-Time PCR System (Applied Biosystems®, Foster City, Calif.) using a standard 40 cycle protocol.

### RPA Reactions:

Reagents for RPA were provided in TwistAmp® exo kit (TwistDx™ Cambridge, UK) and RPA reactions were performed according to the manufacturer's instruction. Briefly, a 47.5 μL mixture containing 29.5 μL rehydration buffer, 300 nM of each primer (Anaplasma RPA_3F/Anaplasma RPA_3R), 120 nM probe and DNA template (2 to 10 μL) was added and mixed with lyophilized RPA enzymes. After adding 2.5 μL of magnesium acetate (MgAc, 280 mM) to start the reaction, the 8-tube reaction strip was immediately mixed and placed in Twista® tube scanner instrument (TwistDx™) for incubation at 39° C. Four minutes after the start of reaction, the strip was quickly removed and vortexed to mix one more time before incubation at 39° C. for another 16 minutes. Fluorescence signal was monitored and analyzed in the Twista® Studio software. The fluorescent probe was designed to provide additional specificity (fluorescence release only occurs after specific binding of the probe to the target sequence) and for convenient real time fluorescent detection of the amplification product.

### Clinical Samples:

Human blood samples and/or DNA from patients with A. phagocytophilum or E. chaffeensis infection were stored frozen at −80° C. until used, and their acquisition and use were approved through human subject protocols at Johns Hopkins Medicine (Baltimore, Md.), University of Maryland, Baltimore, or the St. Mary's/Duluth Clinic (Duluth, Minn.) IRBs. The final diagnosis was identified based on the presence of pathogen DNA in blood by PCR, observation of morulae in circulating leukocytes on acute phase blood smears, by culture, and/or by demonstration of a four-fold increase in specific antibody titer after infection. The samples were blindly tested to reduce any possible bias during the experimentation. Each DNA sample was tested three times and considered as positive with at least 2 out of 3 reactions.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Example 1: Identification of Multicopy Sequences in A. phagocytophilum Genome and RPA Assay Design

Bioinformatics analysis of the A. phagocytophilum (HZ strain) complete genome sequence identified numerous repeated DNA fragments. One of these fragments is within the msp2 gene and has a total of 16 copies. A survey of eight other A. phagocytophilum strains with their complete genome available revealed 12 to 21 copies that share 100% sequence identity (FIG. 1A). BLAST search with other species within Anaplasma genus such as A. marginatle, A. centrale or other closely related species, such as E. chaffeensis, did not result in any significant homology. These data indicate that this 171-bp region is well conserved within strains of A. phagocytophilum, yet highly specific to A. phagocytophilum, making it an ideal target for designing molecular-detection assays. Three forward and three reverse RPA primers were designed and tested with conventional PCR for their performance (FIG. 1B and Table 1). Primers “F3” (SEQ ID NO: 4) and “R3” (SEQ ID NO: 7) were chosen due to high yield of amplicon and a corresponding fluorescent probe was designed (FIG. 1C).

### Example 2: Limit of Detection of the RPA Assay is One Genome Copy of A. phagocytophilum

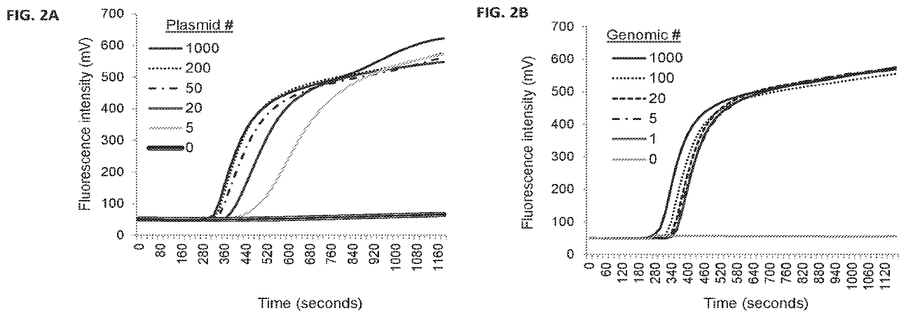

To evaluate the performance of the RPA amplification, a reference plasmid was first generated by inserting a DNA fragment covering the RPA amplicon region. Five to 1000 copies of this plasmid in 10 μL volume were made from serial dilutions. Amplification was detected in all samples containing plasmids and our RPA assay reliably detected the presence of 5 copies of plasmid within 10 minutes of reaction (FIG. 2A). Since A. phagocytophilum Webster strain contains 19 copies of the 171-bp DNA fragment, it was expected that, in theory, the RPA assay would be sensitive enough to detect even less than 1 genome copy of A. phagocytophilum. Indeed, when various genomic copy numbers of A. phagocytophilum were used as template for RPA assay, specific amplification was observed in reactions containing 1000 to as little as 1 genomic copy of A. phagocytophilum DNA (FIG. 2B).

### Example 3: A. phagocytophilum RPA Assay has High Analytical Specificity

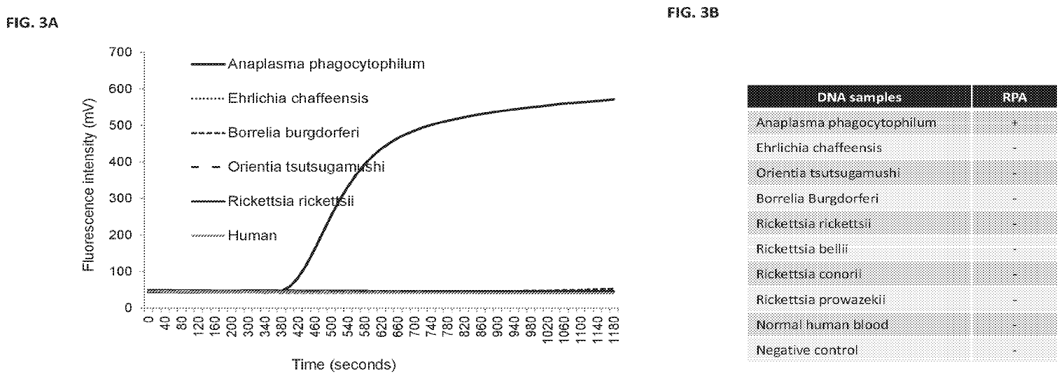

BLAST analysis indicated that the 171-bp DNA sequence did not share significant homology with any other species even within Anaplasma genus; thus, an RPA assay on DNA from a variety of sources was performed for confirmation. As indicated in FIG. 3A and FIG. 3B, no amplification was observed when the following DNA templates were added: E. chaffeensis (Liberty strain, 1×10⁴ copies), B. burgdorferi (B31 strain, 1×10⁵ copies), Orientia tsutsugamushi (Karp strain, 2×10⁴ copies), Rickettsia rickettsia (2×10⁵ copies), Rickettsia bellii (2×10⁵ copies), Rickettsia prowazekii (2×10⁵ copies), Rickettsia conorii (2×10⁵ copies) and human DNA (1×10⁵ copies). These results indicate the A. phagocytophilum RPA assay is highly specific and does not cross-react with human DNA or any closely related bacterial DNA.

### Example 4: A. phagocytophilum RPA Assay has High Analytical and Clinical Sensitivity

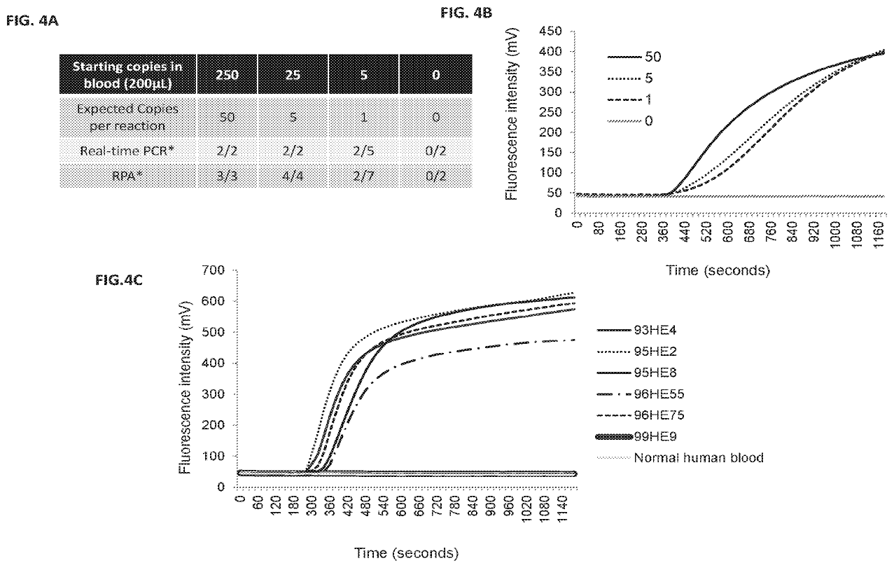

In order to test the sensitivity of our RPA assay for blood samples, clinical patient samples were mimicked by spiking DNA of various genomic copies of A. phagocytophilum into 200 μL of normal human blood. DNA was extracted and eluted into 204 elution buffer and 44 of which was used for each real-time PCR or RPA reaction. As demonstrated in FIG. 4A and FIG. 4B, while 5 copies in 200 μL blood can be detected in 2 out of 7 trials for the RPA assay, 25 copies in 200 μL whole blood resulted in 100% detection rate (4 out of 4 trials). Overall, the performance of our RPA assay was very similar to that of the real-time PCR assay targeting the same 171-bp region in terms of the limit of detection in mimicked clinical samples. These results indicate that the A. phagocytophilum RPA assay could be as sensitive as real time PCR and offer reliable detection of this pathogen in human patients with at least 125 bacteria/mL in whole blood.

The clinical applicability of A. phagocytophilum RPA assay for DNA extracted from blood samples of human patients or healthy blood donors (FIG. 4C and Table 2) was evaluated. For the RPA assay using DNA extracted from clinical blood samples depicted in Table 2, “Anaplasma RPA″=Anaplasma detection by RP As summarized in Table 2, A. phagocytophilum RPA assay was able to identify 100% (8/8) of the patients that were serology and/or PCR-positive at the time of admission. Ehrlichiosis caused by a very closely related bacterium, E. chaffeensis shares similar clinical symptoms and signs of HGA and can yield cross-reactive serologic responses that confound diagnosis. Among the four patients diagnosed as having E. chaffeensis infection, all tested negative by RPA assay, as did six samples from healthy human blood. These data prove that this RPA assay is highly sensitive and specific for detecting A. phagocytophilum in clinical samples.

TABLE 2
RPA assay using DNA extracted from clinical blood samples

| | Anaplasma | Anaplasma | Clinical Test Results | | | |
|---|---|---|---|---|---|---|
| Patient | Detection | Detection | | | Blood | |
| Sample | RPA | qPCR† | Serology | Culture | smear | PCR‡ |
| 01HE5 | Neg | Neg | E. chaffeensis | E. chaffeensis | n.d. | E. chaffeensis |
| 99HE26 | Neg | Neg | E. chaffeensis | E. chaffeensis | Pos | E. chaffeensis |
| 99HE9 | Neg | Neg | acute only: negativ [symbol] | E. chaffeensis | Pos | E. chaffeensis |
| 96HE19 | Neg | Neg | E. chaffeensis | n.d. | n.d. | E. chaffeensis |
| 14HE01 | Neg | Neg | n.d. | Neg | Pos | E. chaffeensis |
| 93HE4 | Pos | Pos | A. phagocytophilu [symbol] | n.d. | Pos | A. phagocytophilu [symbol] |
| 93HE8b | Pos | Pos | A. phagocytophilu [symbol] | n.d. | Pos | A. phagocytophilu [symbol] |
| 95HE2 | Pos | Pos | A. phagocytophilu [symbol] | n.d. | Pos | A. phagocytophilu [symbol] |
| 95HE8 | Pos | Pos | A. phagocytophilu [symbol] | n.d. | Pos | A. phagocytophilu [symbol] |
| 96HE55 | Pos | Pos | A. phagocytophilu [symbol] | A. phagocytophilu [symbol] | Pos | A. phagocytophilu [symbol] |
| 96HE75 | Pos | Pos | A. phagocytophilu [symbol] | n.d. | Pos | A. phagocytophilu [symbol] |
| 06HE3 | Pos | Pos | A. phagocytophilu [symbol] | n.d. | Pos | A. phagocytophilu [symbol] |
| 08HE03 | Pos | Pos | acute only: negativ [symbol] | n.d. | Pos | A. phagocytophilu [symbol] |
| 96HE164 | Pos | Pos | A. phagocytophilu [symbol] | Neg | Pos | A. phagocytophilu [symbol] |
| 96HE165 | Pos | Pos | A. phagocytophilu [symbol] | Neg | Pos | A. phagocytophilu [symbol] |
| 97HE56 | Pos | Pos | A. phagocytophilu [symbol] | Neg | Pos | A. phagocytophilu [symbol] |
| 97HE57 | Pos | Pos | A. phagocytophilu [symbol] | Neg | Pos | A. phagocytophilu [symbol] |
| 97HE97 | Pos | Pos | A. phagocytophilu [symbol] | A. phagocytophilu [symbol] | Pos | A. phagocytophilu [symbol] |
| 98HE4 | Pos | Pos | A. phagocytophilu [symbol] | n.d. | Pos | A. phagocytophilu [symbol] |
| 98HE24 | Pos | Pos | n.d. | n.d. | n.d. | A. phagocytophilu [symbol] |
| 98HE28 | Pos | Pos | negative | n.d. | n.d. | A. phagocytophilu [symbol] |
| 97HE300 | Pos | Pos | A. phagocytophilu [symbol] | n.d. | n.d. | A. phagocytophilu [symbol] |
| E-PCR72 | Pos | Pos | A. phagocytophilu [symbol] | n.d. | n.d. | A. phagocytophilu [symbol] |
| 98HE3 | Pos | Pos | A. phagocytophilu [symbol] | negative | Pos | A. phagocytophilu [symbol] |
| E-PCR51 | Pos | Pos | acute only: negativ [symbol] | n.d. | n.d. | A. phagocytophilu [symbol] |
| 96HE76 | Pos | Pos | A. phagocytophilu [symbol] | A. phagocytophilu [symbol] | Pos | A. phagocytophilu [symbol] |
| 96HE73 | Pos | Pos | A. phagocytophilu [symbol] | n.d. | Pos | A. phagocytophilu [symbol] |
| 96HE74 | Pos | Pos | A. phagocytophilu [symbol] | n.d. | Pos | A. phagocytophilu [symbol] |
| 97HE242 | Pos | Pos | A. phagocytophilu [symbol] | n.d. | n.d. | A. phagocytophilu [symbol] |
| 96HE68 | Pos | Pos | A. phagocytophilu [symbol] | n.d. | n.d. | A. phagocytophilu [symbol] |
| 96HE53 | Pos | Pos | negative | A. phagocytophilu [symbol] | Pos | A. phagocytophilu [symbol] |
| 96HE77 | Pos | Pos | A. phagocytophilu [symbol] | A. phagocytophilu [symbol] | Pos | A. phagocytophilu [symbol] |
| 96HE57 | Pos | Pos | A. phagocytophilu [symbol] | n.d. | n.d. | A. phagocytophilu [symbol] |
| E-PCR91 | Pos | Pos | acute only: negativ [symbol] | n.d. | n.d. | A. phagocytophilu [symbol] |
| 11HE09 | Pos | Neg | acute only: negativ [symbol] | n.d. | n.d. | A. phagocytophilu [symbol] |
| 10HE08 | Pos | Pos | acute only: negativ [symbol] | n.d. | n.d. | A. phagocytophilu [symbol] |
| NHB 2 | Neg | Neg | n.d. | n.d. | n.d. | n.d. |
| NHB 11 | Neg | Neg | n.d. | n.d. | n.d. | n.d. |
| NHB A | Neg | Neg | n.d. | n.d. | n.d. | n.d. |
| NHB B | Neg | Neg | n.d. | n.d. | n.d. | n.d. |
| NHB C | Neg | Neg | n.d. | n.d. | n.d. | n.d. |
| NHB D | Neg | Neg | n.d. | n.d. | n.d. | n.d. |

(pos = positive; neg = negative; n.d. = not determined. † = using primer sets of both
msp2F/msp2R and ApMSP2f/ApMSP2r for A. phagocytophilum detection; ‡ = clinical test
PCR, targeting either 16S rRNA or msp2 genes, was performed at admitting hospitals using
blood samples collected during acute phase of infection). [symbol]
indicates data missing or illegible when filed

It is noted herein that isothermal amplification for A. phagocytophilum was developed recently by Pan et al. using loop-mediated isothermal amplification (LAMP) (Pan et al. 2011 J Clin Microbiol 49:4117-4120). Compared with LAMP (Notomi et al. 2000 DNA Nucleic Acids Res 28: E63), RPA assay is carried out at lower temperatures (37-42° C. vs. 60-65° C.) with less reaction time (20 minutes vs. 60 minutes). The limit of detection for the LAMP assay reported by Pan et al. is 25 copies per reaction using reference plasmids, while the RPA assay disclosed herein is 5 copies. Furthermore, although the same msp2 gene was used for both assays, the region for primer design used by Pan et al. has fewer copies compared with the 171-bp sequence in the RPA assay in genomes from both Webster and HZ strain which is the target of the assay disclosed herein. These differences would be predicted to result in higher sensitivity for the RPA assay when applied to clinical samples. Indeed, the RPA assay of the present application demonstrated 100% sensitivity to detect previously diagnosed A. phagocytophilum clinical cases (8/8). In terms of specificity, RPA reactions using DNA from a wide range of organisms, including human and phylogenetically closely-related bacteria, were analyzed and data provided herein indicate that no cross-reactivity was observed. Additional assays of multiple clinical cases of E. chaffeensis infection were performed and no amplification was observed.

It is contemplated herein that future studies may comprise larger numbers of well-defined clinical cases to further evaluate the methods of the present application for its clinical applicability. That said, data provided herein clearly demonstrate very high sensitivity and specificity may be obtained with the assay of the present application using currently available samples. Indeed, the disclosed methods provide a sensitive assay to detect Anaplasmosis in human or animal samples using a multiple copy target gene and an isothermal amplification. Specifically, the use of the disclosed target gene for amplification according to the methods of the instant present application provides an exceptional sensitivity (Table 2) and the use of isothermal amplification further allows the amplification and detection to be performed in a heating block with the requirement of only 10 minutes to achieve 100% sensitivity and specificity.

All patents, patent applications, and published references cited herein are hereby incorporated by reference in their entirety. While the claimed invention has been particularly shown and described with references to preferred embodiments thereof, it will be understood by those skilled in the art that various changes in form and details may be made therein without departing from the scope of the invention encompassed by the appended claims. The claims are intended to cover the components and steps in any sequence which is effective to meet the objectives there intended, unless the context specifically indicates the contrary.

What is claimed is:

1. A method of detecting the presence of Anaplasma phagocytophilum (A. phagocytophilum) in a sample comprising:
(a) amplifying a 171-base pair (bp) target DNA sequence encoded by SEQ ID NO: 1, or a fragment thereof, within the msp2 gene in the A. phagocytophilum genome; and (b) detecting the amplification products of step (a);
wherein the detection of amplification products in step (b) indicates the presence of A. phagocytophilum in the sample.

2. A method of diagnosing tick-borne fever (TBF) in an animal comprising detecting the presence of A. phagocytophilum in a sample from said animal, said method comprising:
a) amplifying a 171-bp target DNA sequence encoded by SEQ ID NO: 1, or a fragment thereof, within the msp2 gene in the A. phagocytophilum genome; and (b) detecting amplification products of step (a);
wherein the detection of said amplification products indicates a positive diagnosis of TBF in said animal.

3. A method of diagnosing human granulocytic anaplasmosis (HGA) in a human comprising detecting the presence of A. phagocytophilum in a sample from said human, said method comprising:
a) amplifying a 171-bp target DNA sequence encoded by SEQ ID NO: 1, or a fragment thereof, within the msp2 gene in the A. phagocytophilum genome; and (b) detecting amplification products of step (a);
wherein the detection of said amplification products indicates a positive diagnosis of HGA in said human.

4. The method of any one of claims 1-3 wherein the fragment of the 171-bp target DNA sequence is selected from the group consisting of nucleotide fragments ranging from about 50 bp to about 100 bp in length found within SEQ ID NO: 1.

5. The method of any one of claims 1-3 wherein the fragment of the 171-bp target DNA sequence is a nucleotide fragment ranging from about 50 to about 100 bp long found within nucleotides 2 to 151 of SEQ ID NO: 1, or within nucleotides 32 to 170 of SEQ ID NO: 1.

6. The method of any one of claims 1-3 wherein the amplification step (a) comprises the use of a forward primer selected from the group consisting of forward primers designed within nucleotides 2 to 151 of SEQ ID NO: 1 and a reverse primer designed within nucleotides 32 to 170 of SEQ ID NO: 1.

7. The method of claim 6 wherein said forward primer is selected from the group consisting of forward primers encoded by SEQ ID NO: 2, SEQ ID NO: 3, and SEQ ID NO: 4 and wherein said reverse primer is selected from the group consisting of reverse primers encoded by SEQ ID NO: 5, SEQ ID NO: 6, and SEQ ID NO: 7.

8. The method of claim 7 wherein said forward primer is encoded by SEQ ID NO: 4 and wherein said reverse primer is encoded by SEQ ID NO: 7.

9. The method of any one of claims 1-3 wherein the step of detecting amplification products comprises using end-point assays and/or detecting the amplification products in real time.

10. The method of claim 9 wherein said end-point assays comprise gel electrophoresis or sandwich assays.

11. The method of any one of claims 1-3 wherein the step of detecting amplification products comprises using fluorescence.

12. The method of any one of claims 1-3 wherein the step of detecting amplification products comprises using one or more probes.

13. The method of claim 12 wherein said one or more probes are selected from the group consisting of fluorescent probes, non-fluorescent probes, and antigenically labeled probes.

14. The method of claim 12 wherein said one or more probes are nucleic acid probes.

15. The method of claim 14 wherein said nucleic acid probe is a nucleic acid probe encoded by SEQ ID NO: 8.

16. The method of claim 13 wherein said antigenically labeled probes are selected from the group consisting of fluoroscein-, digoxigenin-, and biotin-labeled probes.

17. The method of claim 12 wherein said one or more probes are labeled with a reporter fluorophore at the 5′ end of the probe sequence and a quencher fluorophore at the 3′ end of the probe sequence.

18. The method of claim 17 wherein the reporter fluorophore is selected from the group consisting of fluoroscein, fluorescein isothiocyantate (FITC), 6-carboxy-2′,4,4′,5′,7,7′-hexachlorofluorescein, 6-carboxy-4′,5′-dichloro-2′,7′-dimethoxyfluorescein succinimidyl ester, and tetrachlorofluorescein and the quencher fluorophore is selected from the group consisting of fluorescent and non-fluorescent quenchers.

19. The method of any one of claims 1-3 wherein the step of detecting amplification products comprises using immunochromatography.

20. The method of claim 19 wherein said immunochromatography comprises the use of a lateral flow immunoassay.

21. The method of claim 1 wherein said sample is a human or animal sample.

22. The method of claim 2 or claim 21 wherein said animal is an equine.

23. The method of any one of claims 1-3 wherein said amplification step (a) comprises the use of an isothermal amplification reaction.

24. The method of claim 23 wherein said isothermal amplification reaction is a recombinase polymerase amplification reaction (RPA).

25. A kit comprising one or more forward primers selected from the group consisting of forward primers designed within nucleotides 2 to 151 of SEQ ID NO: 1 and one or more reverse primers designed within nucleotides 32 to 170 of SEQ ID NO: 1, wherein said kit may be used for detecting the presence of A. phagocytophilum in a sample and/or for diagnosing TBF in an animal and/or for diagnosing HGA in a human comprising using a nucleic acid amplification reaction.

26. The kit of claim 25 wherein the one or more forward primers is selected from the group consisting of forward primers encoded by SEQ ID NO: 2, SEQ ID NO: 3, and SEQ ID NO: 4 and wherein the one or more reverse primers is selected from the group consisting of reverse primers encoded by SEQ ID NO: 5, SEQ ID NO: 6, and SEQ ID NO: 7.

27. The kit of claim 26 wherein the forward primer is encoded by SEQ ID NO:4 and wherein the reverse primer is encoded by SEQ ID NO: 7.

28. The kit of claim 25 further comprising one or more additional reagents for performing the nucleic acid amplification reaction.

29. The kit of claim 25 wherein said nucleic acid amplification reaction is an isothermal amplification reaction.

30. The kit of claim 29 wherein the isothermal amplification reaction is a recombinase polymerase amplification.

31. The kit of claim 25 further comprising one or more reagents for detecting an amplicon of the nucleic acid amplification reaction.

32. The kit of claim 31 wherein said one or more reagents for detecting the amplicon are used for detecting the amplicon using endpoint assays and/or for detecting the amplicon in real time.

33. The kit of claim 31 wherein said one or more reagents for detecting the amplicon are used for detecting the amplicon using agarose gel electrophoresis and/or using immunochromatography.

34. A method of treating a pathological condition caused by A. phagocytophilum in a subject in need thereof comprising
(a) administering to said subject a therapeutically effective amount of an anti-A. phagocytophilum agent, wherein prior to the administering step the presence of A. phagocytophilum is detected in a sample from the subject according to any one of methods 1-23.

35. A method of treating a pathological condition caused by A. phagocytophilum in a subject in need thereof comprising
(a) obtaining a biological sample from said subject;
(b) detecting the presence of A. phagocytophilum in said biological sample from said subject by amplifying a 171-bp target DNA sequence encoded by SEQ ID NO: 1, or a fragment thereof, within the msp2 gene in the A. phagocytophilum genome and detecting the amplification products, wherein the detection of said amplification products indicates a positive diagnosis of said pathological condition caused by A. phagocytophilum in said subject; and
(c) administering to said subject a therapeutically effective amount of an anti-A. phagocytophilum agent.

36. The method of claim 34 or 35 wherein said subject is a human and said pathological condition is HGA.

37. The method of claim 34 or 35 wherein said subject is an animal and said pathological condition is TBF.

38. The method of claim 37 wherein said animal is a horse.

39. The method of claim 34 or 35 wherein said positive diagnosis is made within one week after infection by A. phagocytophilum.

40. The method of claim 34 or 35 wherein said biological sample is a blood sample.

41. The method of claim 34 or 35 wherein said anti-A. phagocytophilum agent is an antibiotic.

42. The method of claim 41 wherein said antibiotic is doxycycline.


SEQUENCE LISTING (14 sequences)

SEQ ID NO: 1 - DNA, 171 bp, Artificial Sequence
  Synthetic sequence
tagatatactgtatcagcttcatcttccttactaccactatctctaatacccttggtcttgaagcgctcgtaaccaatctcaagctcaaccctggcaccaccaataccataaccaacactgccttccatagctacaagcatgttgtccttaaacccaatccgaggatcagg

SEQ ID NO: 2 - DNA, 30 bp, Artificial Sequence
  Synthetic sequence
tctaatacccttggtcttgaagcgctcgta

SEQ ID NO: 3 - DNA, 30 bp, Artificial Sequence
  Synthetic
tggtcttgaagcgctcgtaaccaatctcaa

SEQ ID NO: 4 - DNA, 30 bp, Artificial Sequence
  Synthetic
ctcgtaaccaatctcaagctcaaccctggc

SEQ ID NO: 5 - DNA, 30 bp, Artificial Sequence
  Synthetic
catgcttgtagctatggaaggcagtgttgg

SEQ ID NO: 6 - DNA, 30 bp, Artificial Sequence
  Synthetic
ctgatcctcggattgggtttaaggacaaca

SEQ ID NO: 7 - DNA, 30 bp, Artificial Sequence
  Synthetic
tcctcggattgggtttaaggacaacatgct

SEQ ID NO: 8 - DNA, 50 bp, Artificial Sequence
  Synthetic
  misc_feature (32)..(32): “n” is tetrahydrofuran
aatctcaagctcaaccctggcaccaccaatanataaccaacactgccttc

SEQ ID NO: 9 - DNA, 20 bp, Artificial Sequence
  Synthetic
gtcttgaagcgctcgtaacc

SEQ ID NO: 10 - DNA, 22 bp, Artificial Sequence
  Synthetic
gcttgtagctatggaaggcagt

SEQ ID NO: 11 - DNA, 25 bp, Artificial Sequence
  Synthetic
cagtcgtgaatgtagagggaaaaac

SEQ ID NO: 12 - DNA, 22 bp, Artificial Sequence
  Synthetic
ggaatcccccttcaggaacttg

SEQ ID NO: 13 - DNA, 27 bp, Artificial Sequence
  Synthetic
atggaaggtagtgttggttatggtatt

SEQ ID NO: 14 - DNA, 20 bp, Artificial Sequence
  Synthetic
ttggtcttgaagcgctcgta